(19) United States
(12) Patent Application Publication
(10) Pub. No.: US 2022/0143594 A1
(43) Pub. Date: May 12, 2022

(54) CELL TRAPPING ARRAYS WITH SELECTIVE EJECTION

(71) Applicant: Hewlett-Packard Development Company, L.P., Spring, TX (US)

(72) Inventors: Alexander Govyadinov, Corvallis, OR (US); Viktor Shkolnikov, Palo Alto, CA (US)

(73) Assignee: Hewlett-Packard Development Company, L.P., Spring, TX (US)

(21) Appl. No.: 17/415,161

(22) PCT Filed: Jul. 25, 2019
(86) PCT No.: PCT/US2019/043395

§ 371 Date: Jun. 17, 2021

(51) Int. Cl. B01L 3/02; B01L 3/00; G01N 15/14

(52) U.S. Cl. CPC B01L 3/0268; B01L 3/50273; B01L 3/502761; B01L 2300/0864; B01L 2400/0442; B01L 2200/0652; B01L 2200/0668; G01N 15/1404

(57) ABSTRACT

In example implementations, a cell trapping array is provided. The cell trapping array includes a plurality of plates coupled along adjacent edges to form a channel. A plurality of orifices are formed in a first plate of the plurality of plates of the channel. The plurality of orifices is shaped to create a meniscus of a fluid in the channel in the plurality of orifices that is to attract a single cell from cells flowing through the channel in the fluid. The cell trapping array includes a selective ejection system coupled to a second plate located opposite the first plate of the channel. The selective ejection system is to selectively eject the single cell from one of the plurality of orifices.

### BACKGROUND

Cells can be analyzed for various research and medical reasons. Cell concentrations can be separated such that single cells can be analyzed. Single cell analysis becomes more attractive as the cost of detailed genetic analysis decreases via systems such as multiplexed polymerase chain reaction (PCR), sequencing, and the like. However, the bottleneck in the single cell analysis workflow may arise during cell portioning where individual cells are separated from a cell concentration for analysis.

### BRIEF DESCRIPTION OF THE DRAWINGS

FIG. 1 is a block diagram of a cross-sectional view of an example cell trapping array with selective ejection of the present disclosure;

FIG. 2 is a block diagram of a cross-sectional view of an example cell trapping array with a thermal inkjet (TIJ) resistor array to provide the selective ejection of the present disclosure;

FIG. 3 is a block diagram of a cross-sectional view of the example cell trapping array with the TIJ resistor array and electrodes of the present disclosure;

FIG. 4 is a block diagram of a top view of an example cell trapping array with recirculation flow of the present disclosure;

FIG. 5 is a block diagram of a top view of an example cell trapping array with barriers of the present disclosure;

FIG. 6 is a block diagram of a cross-sectional view of selective ejection of cells in the cell trapping array;

FIG. 7 is a block diagram of a cross-sectional view of in-situ cell staining using a stain dispenser and the cell trapping array with selective ejection of the present disclosure;

FIG. 8 is a block diagram of a cross-sectional view of in-situ cell staining using a stain reagents globally in the cell trapping array with the selective ejection of the present disclosure; and

FIG. 9 is a flow chart of an example method for selectively ejecting cells in a cell trapping array of the present disclosure.

### DETAILED DESCRIPTION

Examples described herein provide a cell trapping array with selective ejection. As noted above, separating cell concentrations into single cells for analysis can be a bottle neck for single cell analysis. Some methods use centrifugation. However, centrifugation solutions are poorly amenable to automation as they are generally difficult to integrate into general microfluidic solutions. The centrifugation solutions may also use expensive equipment associated with the high g-force centrifuges. Other automated methods may require expensive and complicated electronics such as fluorescence activated cell sorting, flow cytometry/fluorescence-activated cell sorting, microfluidic devices that harness acoustic waves, and the like. In addition, these other automated methods may not provide selective ejection of the cells from particular wells after the cells are trapped.

Examples herein provide a cell trapping array that includes a selective ejection system. The cell trapping array may trap cells into an orifice using a “cheerio's effect” to trap cells against a wall of the orifice. The orifices may be sized to trap a desired cell type based on cell size from different cells in a cell reservoir.

Once the cells are trapped in the appropriate orifices, the selective ejection system may allow the cells to be selectively ejected from a desired orifice. For example, the selective ejection system may be a thermal inkjet (TIJ) resistor array.

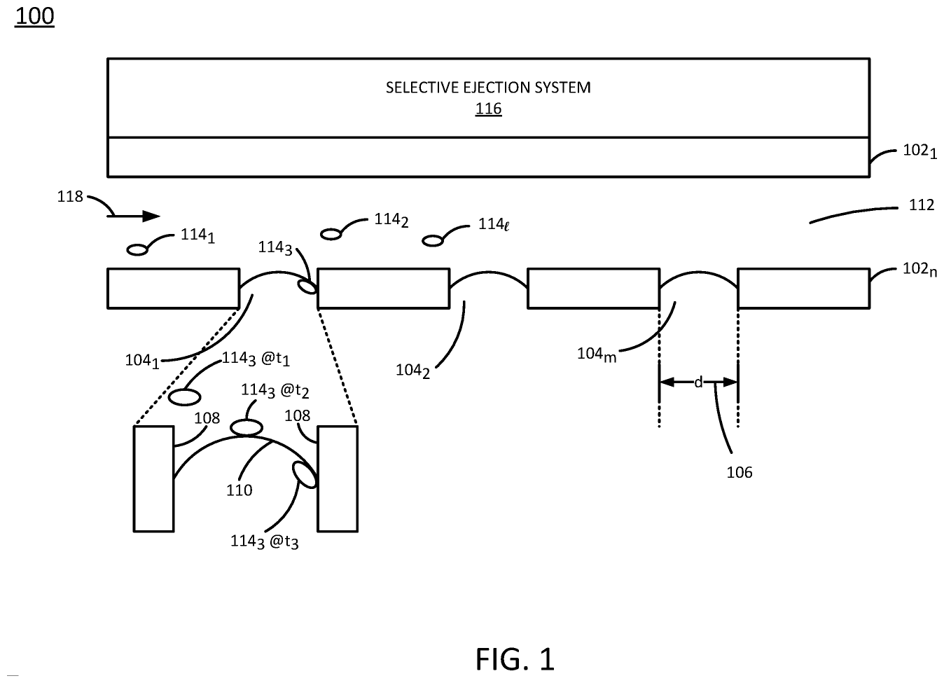

FIG. 1 illustrates a cross-sectional view of an example cell trapping array 100 of the present disclosure. In one example, the cell trapping array 100 includes a plurality of plates 102₁ to 102ₙ (hereinafter also referred to individually as a plate 102 or collectively as plates 102) that are coupled along adjacent edges to form a channel 112. The plates 102 may be fabricated from plastic or metal.

In one example, the plates 102 may be coupled to form the channel 112 with any shaped opening or cross-sectional shape. For example, when looking at the opening of the channel 112, the cross-sectional area of the channel 112 may have a square shape, a rectangular shape, a circular shape, a polygon shape, an irregular shape, and the like.

A plurality of cells 114₁-114ₗ (hereinafter also referred to individually as a cell 114 or collectively as cells 114) may be fed through the channel 112 in a fluid. In other words, the fluid may contain a concentration of the cells 114. The cells 114 may be the same size or may vary in size.

In one example, one of the plates 102 (e.g., the plate 102ₙ in FIG. 1) may include a plurality of orifices 104₁-104ₘ (hereinafter also referred to individually as an orifice 104 or collectively as orifices 104). The orifices 104 may have any shape, such as a circular opening, an oval opening, a slit shape, an irregular shape, and the like.

In one example, each orifice 104 may have a diameter 106 as shown by the line “d” in FIG. 1. In one example, the diameter 106 may be a function of a size of the cell that is to be trapped in a respective orifice 104. For example, if all of the orifices 104 are to trap the same size cell, then the orifices 104 may have the same sized diameter. If the orifices 104 are to trap different size cells, then the orifices 104 may have different sized diameters. In one example, the diameter of the orifices 104 may be approximately 10 to 100 microns.

In one example, the cells 114 may be fed through the channel 112 in the fluid as shown by an arrow 118. As fluid evaporates, the cells 114 may be moved towards the direction of evaporation or towards the orifices 104. The cells 114 may be trapped in, collected in, or attracted to the orifices 104 via the “cheerio's effect.” In one example, each orifice 104 may trap a single cell 114 at a time.

FIG. 1 illustrates a larger view of the orifice 104₁ to illustrate how the “cheerio's effect” works to attract a cell 114₃. The orifice 104₁ may cause the fluid to form a meniscus 110 against walls 108 of the orifice 104₁. The meniscus 110 may have a concave or convex shape. At time t₁, the cell 114₃ may be flowing in the channel 112. As the fluid evaporates, the evaporation of the fluid may drive the cell 114₃ towards the orifice 104₁ at time t₂. When the cell 114₃ encounters the meniscus 110, capillary forces of the meniscus 110 may pull the cell 114₃ towards the immobile part of the meniscus 110 (e.g., the portion of the meniscus 110 against the wall 108). At time t₃, the cell 114₃ may be trapped or held against the wall 108 of the orifice 104₁ via the “cheerio's effect”.

In one example, the cell trapping array 100 may also include a selective ejection system 116. The selective ejection system 116 may apply a force to a selected orifice 104 to eject the cell 114 trapped in the selected orifice 104. The force may be a shockwave or a vibration through the fluid. The force may be large enough to overcome the “cheerio's effect” and eject the cell 114 out of the selected orifice 104.

In other words, the cell trapping array 100 of the present disclosure may selectively eject cells 114 for further analysis, unlike other cell trapping arrays that eject all of the cells at the same time. As discussed in further details below, the selected cell 114 may be ejected into another fluid for further analysis.

In one example, the selective ejection system 116 may be controlled by a controller or a processor (not shown). An orifice 104 may be selected via a user interface (not shown) and the controller may cause the selective ejection system 116 to apply a force towards the selected orifice 104.

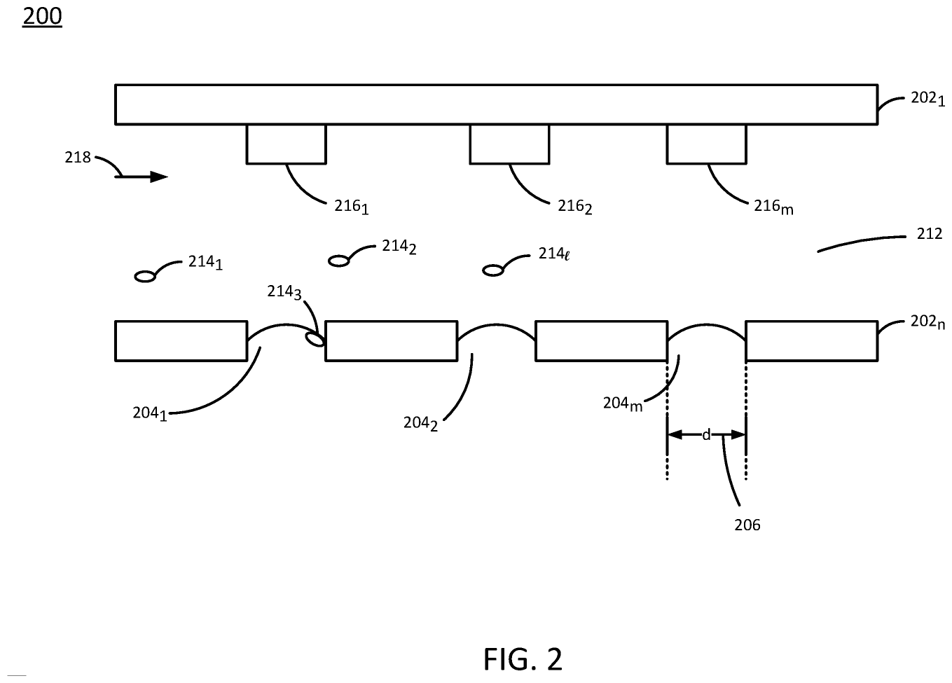

FIG. 2 illustrates a cross-sectional view of a cell trapping array 200. The cell-trapping array 200 may be similar to the cell-trapping array 100. However, the cell trapping array 200 may include thermal inkjet (TIJ) resistors 216₁ to 216ₘ (hereinafter also referred to individually as a TIJ resistor 216 or collectively as TIJ resistors 216).

In one example, the cell trapping array 200 may include plates 202₁ to 202ₙ (hereinafter also referred to individually as a plate 202 or collectively as plates 202) that are coupled along adjacent edges to form a channel 212. The plates 202 may be fabricated from plastic or metal.

In one example, the plates 202 may be coupled to form the channel 212 with any shaped opening or cross-sectional shape. For example, when looking at the opening of the channel 212, the cross-sectional area of the channel 212 may have a square shape, a rectangular shape, a circular shape, a polygon shape, an irregular shape, and the like.

A plurality of cells 214₁-214ₗ (hereinafter also referred to individually as a cell 214 or collectively as cells 214) may be fed through the channel 212 in a fluid. In other words, the fluid may contain a concentration of the cells 214. The cells 214 may be the same size or may vary in size.

In one example, one of the plates 202 (e.g., the plate 202ₙ in FIG. 2) may include a plurality of orifices 204₁-204ₘ (hereinafter also referred to individually as an orifice 204 or collectively as orifices 204). The orifices 204 may have any shape, such as, a circular opening, an oval opening, a slit shape, an irregular shape, and the like.

In one example, each orifice 204 may have a diameter 206 as shown by the line “d” in FIG. 1. In one example, the diameter 206 may be a function of a size of the cell that is to be trapped in a respective orifice 204. For example, if all of the orifices 204 are to trap the same size cell, then the orifices 204 may have the same sized diameter. If the orifices 104 are to trap different size cells, then the orifices 204 may have different sized diameters. In one example, the diameter of the orifices 204 may be approximately 10 to 100 microns.

In one example, the cells 214 may be fed through the channel 212 in the fluid as shown by an arrow 218. As fluid evaporates, the cells 214 may be moved towards the direction of evaporation or towards the orifices 204. The cells 214 may be trapped in, collected in, or attracted to the orifices 204 via the “cheerio's effect,” as illustrated in FIG. 1 and described above.

The cell trapping array 200 may include a plurality of TIJ resistors 216. In one example, each orifice 204 may have a corresponding TIJ resistor 216. Thus, if there are ten orifices 204, then there may be ten TIJ resistors 216. A TIJ resistor 216 may be located above or below, or aligned with, each orifice 204. Said another way, the TIJ resistor 216 may be located to push the cell 214 towards the respective orifice 204 and also to eject the cell 214 out of the orifice 204.

In one example, the TIJ resistor 216 may include a controllable circuit that includes a resistor heater. When the circuit is activated, current may flow through the resistor heater to generate heat. The heat may create a steam bubble in the fluid around the TIJ resistor 216. The steam bubble may move towards the orifice 204. Thus, when a cell 214 is located between the TIJ resistor 216 and the orifice 204, the steam bubble created by the activated TIJ resistor 216 may push the cell 214 towards the orifice 204. The TIJ resistor 216 may help to move the cell 214 in addition to the movement caused by the evaporation of the fluid in the channel 212.

In one example, TIJ resistor 216 may also selectively eject the cell 214 from a selected orifice 204. For example, the cell 214₃ may be trapped in the orifice 204₁. The cell 214₃ may be selected to be ejected for further analysis. Thus, the TIJ resistor 216₁ over the orifice 204₁ may be activated to eject the cell 214₃ from the orifice 204₁. Activation of the TIJ resistor 216₁ may create the steam bubble in the fluid, as described above. However, the steam bubble may burst when it reaches the surface of the fluid in the orifice 204₁. The force created when the steam bubble bursts, may cause the cell 214₃ to be ejected from the orifice 204₁.

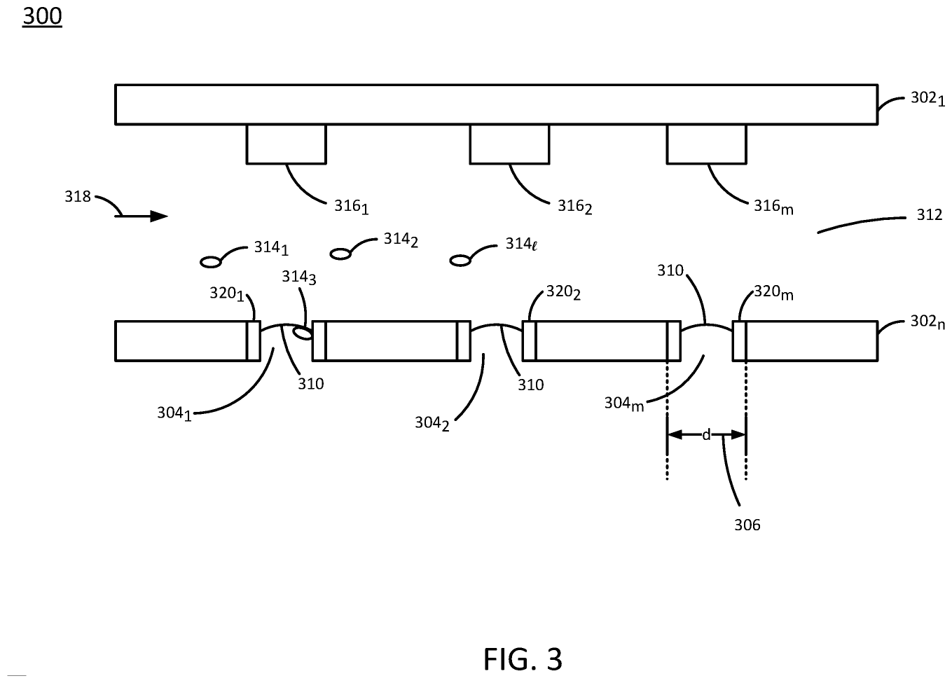

FIG. 3 illustrates a block diagram of a cross-sectional view of a cell trapping array 300 with TIJ resistors 316 and electrodes 320. In one example, the cell trapping array 300 may include a plurality of plates 302₁ to 302ₙ (hereinafter also referred to individually as a plate 302 or collectively as plates 302) to form a channel 312, similar to the cell trapping array 200. A plurality of cells 314₁ to 314ₗ may flow through the channel 312 in a fluid in a direction as shown by an arrow 318. The cell trapping array may include a plurality of TIJ resistors 316₁ to 316ₘ to selective eject the cells 314 from the selected orifices 304₁ to 304ₘ (also referred to herein individually as an orifice 304 or collectively as orifices 304). The cell trapping array 300 may be similar to the cell trapping array 200 except that the cell trapping array 300 may include electrodes 320₁ to 320ₘ (also referred to herein individually as an electrode 320 or collectively as electrodes 320).

In one example, each orifice 304 may include a respective electrode 320₁ to 320ₘ. The electrode 320 may be located around the inner walls of the orifice 304. In one example, the electrodes 320 may be flat as deposited on the surface of the plate 302 around the orifices 304. In one example, the electrode 320 may be a single continuous piece around the inner wall of the orifice 304. In another example, the electrode 320 may include multiple pieces that are located on the inner wall of the orifice 304.

An electrode 320 may be activated to control a size and shape of a meniscus 310 formed in an orifice 304. For example, when an electrode 320 is activated, a voltage may be applied to the electrode 320. The amount of applied voltage may change a wetting angle and a corresponding shape of the meniscus 310. Based upon the amount of applied voltage, the amount of curvature formed in the meniscus 310 may increase or decrease. The change in the shape of the meniscus 310 may control a rate of capture of the cells 314 in the channel 312.

In one example, the electrodes 320 in each orifice 304 may be controlled independently. Thus, the rate of capture for the orifice 304₂ may be increased and the rate of capture for the orifice 304₁ may be decreased. For example, the orifice 304₂ may be sized to capture a cell 314₂ having a first size and the orifice 304₁ may be sized to capture a cell 314₁ having a second, different size. The electrode 320₁ in the orifice 304₁ may be activated to increase the rate of capture of the cell 314₁. However, the electrode 320₂ in the orifice 304₂ may remain deactivated to maintain a rate of capture of the cell 314₂ in the orifice 304₂.

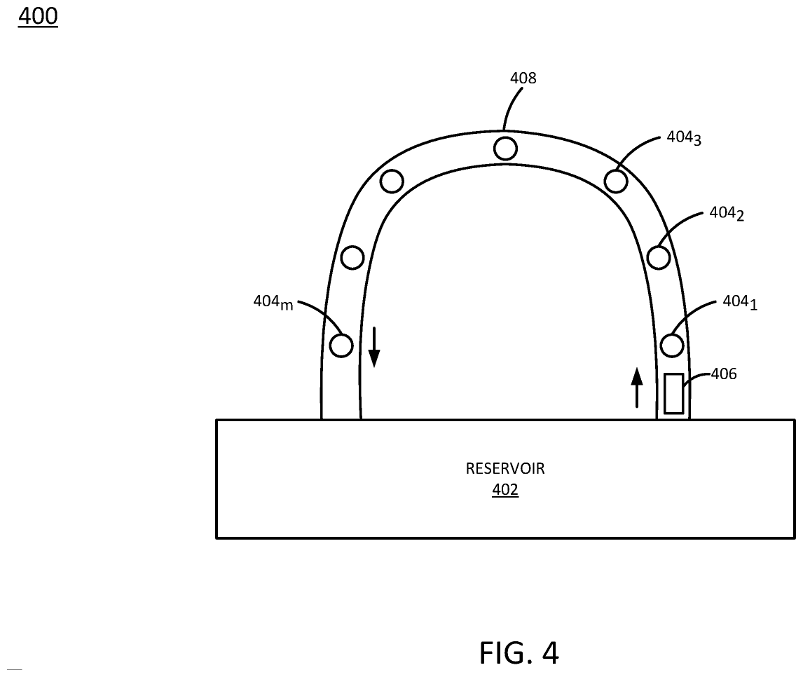

FIG. 4 illustrates a top view of an example cell trapping array 400 with a recirculation flow. In one example, the cell trapping array 400 may include a reservoir 402 that contains the cells in a fluid as illustrated in FIGS. 1-3. Although a single reservoir 402 is illustrated in FIG. 4, it should be noted that multiple reservoirs 402 may be deployed depending on an arrangement of the recirculation loops of the cell trapping array 400.

In one example, the cell trapping array 400 may include a pump 406. The pump 406 may draw the fluid with the cell concentration out of the reservoir 402 and pump the fluid through a recirculation loop 408 as shown by the arrows. The recirculation loop 408 may include a plurality of orifices 404₁ to 404ₘ (hereinafter also referred to individually as an orifice 404 or collectively as orifices 404). Although a plurality of orifices 404 are illustrated in FIG. 4, it should be noted that the cell trap array 400 may have any number of orifices 404 from a single orifice 404 to multiple orifices 404. The size and shape of the orifices 404 may be similar to the orifices illustrated in FIGS. 1-3 and described above.

In one example, the cell trapping array may include a selective ejection system 116 or the TIJ resistors 216, as described above. The pump 406 may pump the fluid through the recirculation loop 408 until a desired number of the cells are trapped or removed from a plurality of different cells in the fluid. For example, the cells may be trapped and selectively ejected as the pump is circulating the fluid through the recirculation loop 408.

In one example, an optical analyzer or sensor may be coupled to the reservoir 402 to measure the cell concentration. When the desired number of cells have been removed from the cell concentration (e.g., trapped in the orifices 404), the pump 406 may be stopped.

Although one example arrangement is illustrated in FIG. 4, it should be noted that the cell trap array 400 may have other arrangements. For example, instead of a single recirculation loop 408, there may be multiple nested recirculation loops 408 with respective pumps 406. Each recirculation loop may be used to trap different sized or types of cells within a plurality of cells in the reservoir 402.

In one example, the recirculation loop 408 may have different shapes. For example, there may be multiple turns or curves in the recirculation loop 408. In one example, the recirculation loop 408 may be connected by different reservoirs 402 on each end. A pump 406 may be located on each end. Thus, a first pump 406 on one end may pump the fluid in a first direction from the first reservoir 402, through the recirculation loop 408, and towards the second reservoir 402. The second pump 406 on the opposite end may then pump the fluid in the opposite direction from the second reservoir 402, through the recirculation loop 408, and back towards the first reservoir 402.

In one example, the pump 406 may be a reversible pump or a two-way pump. Thus, the pump 406 may send the fluid through the recirculation loop 408 in a first direction and then send the fluid through the recirculation loop 408 in the opposite direction.

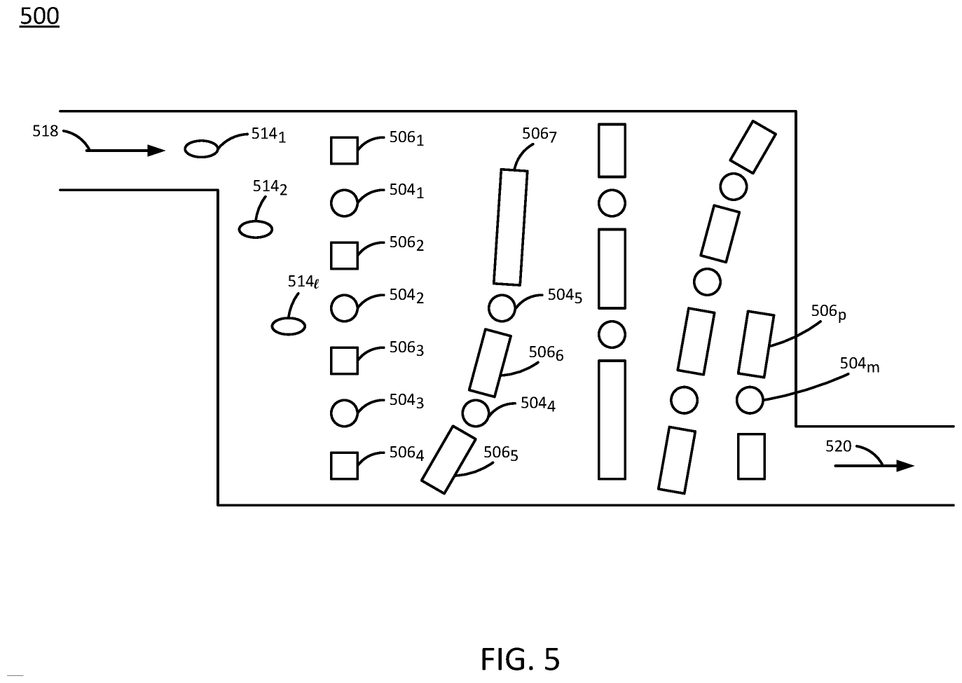

FIG. 5 illustrates a top view of an example cell trapping array 500 with barriers 506. In one example, the fluid with the cell concentration may enter the cell trapping array 500 as shown by an arrow 518. The cell trapping array 500 may include a plurality of orifices 504₁ to 504ₘ (hereinafter also referred to individually as an orifice 504 or collectively as orifices 504). The size and shape of the orifices 504 may be similar to the orifices illustrated in FIGS. 1-3 and described above. In one example, the cell trapping array may include a selective ejection system 116 or the TIJ resistors 216, as described above.

In one example, the cell trapping array 500 may include barriers 506₁ to 506ₚ (hereinafter also referred to individually as a barrier 506 or collectively as barriers 506). The barriers 506 may be used inside of the channel of the cell trapping array 500 to help guide, direct, or navigate cells 514₁-514ₗ towards an orifice 504. Thus, the barriers 506 may increase the probability of the cells 514 being trapped by an orifice 504, which may help to increase the capture rate or efficiency of the cell trapping array 500.

In one example, a barrier 506 may be a wall or a segment that impedes the progress of the cells 514. For example, a barrier 506 may be a rectangular portion that is fitted inside of the channel and between the top and bottom plate of the cell trapping array 500.

In one example, the orifices 504 may be arranged in a line or array. As shown in FIG. 5, the orifices 504₁-504₃ may be arranged in a line. Additional orifices 504 may be arranged in a line to form a square or rectangular array. The corresponding barriers 506₁ to 506₄ may be fitted between the orifices 504₁-504₃ to guide the cells 514 towards the orifices 504₁-504₃.

In one example, the orifices 504 may be arranged more densely in the plate. For example, the orifices 504₄-505ₘ may be arranged randomly or in a “Y” pattern to increase the capture efficiency of the cell trapping array 500. In one example, the corresponding barriers 506₅ to 506ₚ may be fitted between the orifices 504₄-505ₘ to guide the cells 514 towards the orifices 504₄-505ₘ. In one example, the orifices 504 may be all arranged in a square array as shown by the orifices 504₁-504₃, may be all arranged in a dense arrangement as shown by the orifices 504₄-505ₘ, or a combination of both.

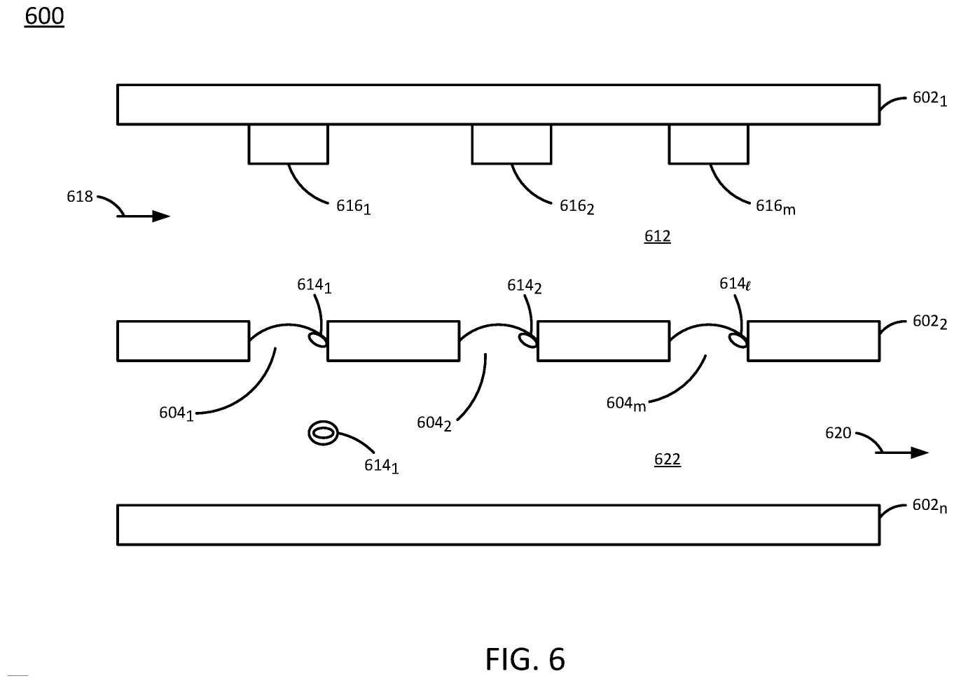

FIG. 6 illustrates a cross-sectional view of selective ejection of cells in a cell trapping array 600. In one example, the cell trapping array 600 may include channels 612 and 622 formed by plates 602₁ to 602ₙ. A fluid with cells 614₁ to 614ₗ may be fed through the channel 612 as shown by an arrow 618. A plurality of orifices 604₁ to 604ₘ may be formed in a plate 602₂. The cell trapping array 600 may also include a plurality of TIJ resistors 616₁ to 616ₘ located on a plate 602₁ that is opposite the plate 602₂ with the orifices 604.

In one example, the orifices 604 may be similar to the orifices illustrated in FIGS. 1-3 and described above. The orifices 604 may trap the cells 614 via the “cheerio's effect” as illustrated in FIG. 1 and described above.

As noted above, the TIJ resistors 616 may selectively eject a cell 614 from a selected orifice 604. The cell 614 may be ejected for further analysis. In one example, the cell trapping array 600 may include the second channel 622 that may include a second fluid that is different than the first fluid. For example, the second fluid may be any fluid that is immiscible with the first fluid. The interfacial surface tension of the fluids may prevent the fluids from mixing. For example, the first fluid in the channel 612 may be water and the second fluid in the second channel 622 may be oil.

Although the fluid in the second channel 622 is illustrated as being in direct contact with the fluid in the first channel 612, it should be noted that the fluids may be separated. For example, an air interface may be deployed between the fluid in the first channel 612 and the fluid in the second channel 622.

In one example, the cell 614₁ may be trapped in the orifice 604₁. The cell 614₁ may be selected for further analysis. As a result, the TIJ resistor 616₁ may be selected to fire to eject the cell 614₁ from the orifice 604₁, as described above. The cell 614₁ may then be ejected into the fluid in the channel 622 to form an emulsion for further analysis, as shown in FIG. 6.

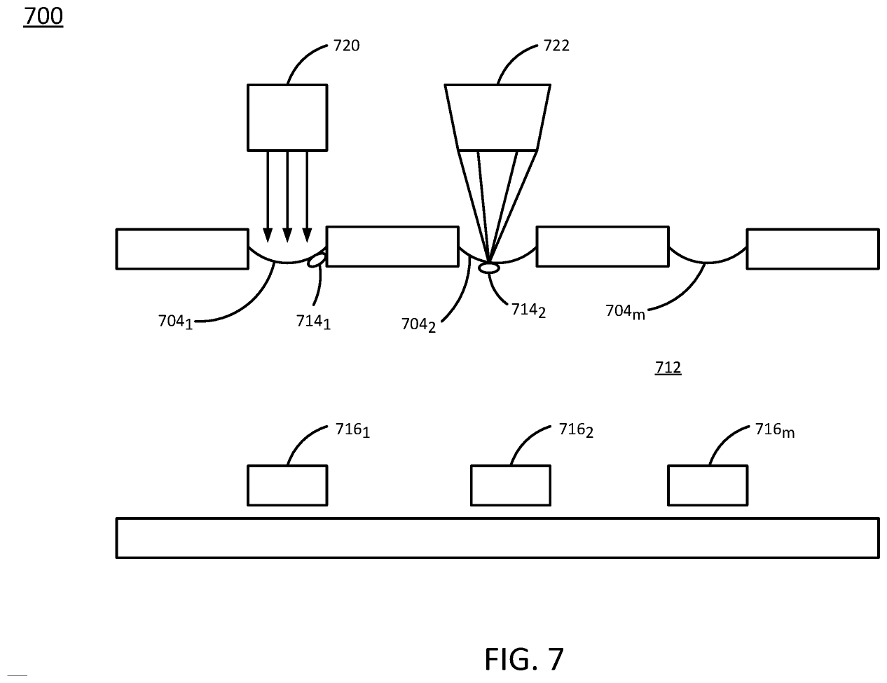

FIG. 7 illustrates a block diagram of a cross-sectional view of in-situ cell staining using a cell trapping array 700 of the present disclosure. In one example, the cell trapping array 700 may include a channel 712. A concentration of cells 714₁ and 714₂ in a fluid may be fed through the channel 712.

In one example, the channel 712 may include orifices 704₁ to 704ₘ. The orifices 704₁ to 704ₘ may be similar to the orifices illustrated in FIGS. 1-3 and described above. The cell trapping array 700 may include TIJ resistors 716₁-716ₘ. The TIJ resistors 716 may operate similar to the TIJ resistors illustrated in FIGS. 2 and 3 and described above.

In one example, the cell trapping array 700 may include a dispenser 720 and an optical analyzer 722. The dispenser 720 may be a multi-well dispenser that can dispense different types of staining reagents. For example, based on a type of cell 714 that is trapped in an orifice 704, a particular type of staining reagent may be dispensed by the dispenser 720. The dispenser 720 may jet the staining reagent towards the trapped cell 714.

In another example, different staining reagents may be jetted into the same cell 714 for different types of analysis. For example, different staining reagents may be jetted into the cell 714 to see which staining reagents react with which portions of the cell 714. The staining reagent may be any type of reagent such as a Gram stain for bacteria identification or a specific antibody stain (e.g., CD45) for cell identification.

The optical analyzer 722 may then be used to analyze the stained cell 714. The optical analyzer 722 may be an illuminated focal microscope. The optical analyzer 722 may capture images of the stained cell 714 to analyze the structures inside of the cell 714 that have been stained.

Although illustrated as separate components, the dispenser 720 and the optical analyzer 722 may be part of a single structure or component. In one example, the dispenser 720 and the optical analyzer 722 may be on a movable track or rail to allow the dispenser 720 and the optical analyzer 722 to move between different orifices 704. Thus, the cell trapping array 700 may allow for in-situ staining and analysis of the cells 714.

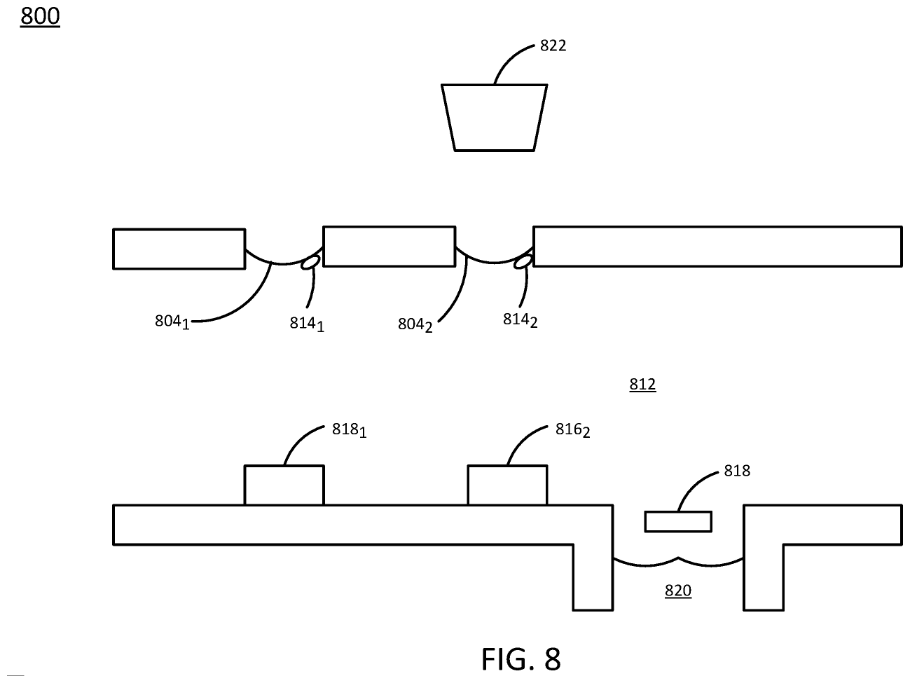

FIG. 8 illustrates a block diagram of a cross-sectional view of in-situ cell staining using a cell trapping array 800. The cell trapping array 800 may include a channel 812. A concentration of cells 814₁ and 814₂ in a fluid may be fed through the channel 812.

In one example, the channel 812 may include orifices 804₁ and 804₂. The orifices 804₁ and 804₂ may be similar to the orifices illustrated in FIGS. 1-3 and described above. The cell trapping array 800 may include TIJ resistors 816₁ and 816₂. The TIJ resistors 816 may operate similar to the TIJ resistors illustrated in FIGS. 2 and 3 and described above.

In one example, the cell trapping array 800 may globally introduce a staining reagent 820 located in a side channel. For example, the staining reagent 820 may be stored in a reservoir and injected into the fluid via the side channel. The staining reagent may be mixed with the fluid to globally stain the cells 814. The staining reagent 820 may be injected by activating a TIJ based inertial pump 818.

In one example, additional side channels may be located downstream to globally the stain the cells 814 as the cells 814 move down the channel 812. For example, cells 814 trapped in the orifices 804 of a first portion of the cell trapping array 800 may be globally stained with a first staining reagent 820. The cells 814 may be analyzed and then ejected to flow further down the channel 812 and be trapped by a second set of orifices 804 in a second portion of the cell trapping array 800. A second side channel may inject a second staining reagent 820 to globally stain the cells 814 a second time, and so forth. The staining reagent 820 may be any type of reagent such as a Gram stain for bacteria identification or a specific antibody stain (e.g., CD45) for cell identification.

In one example, the TIJ resistors 816 may be activated to help mix the staining reagent 820 into the fluid to stain the cells 814. For example, the TIJ resistors 816 can be activated to create steam bubbles that can burst and cause the fluid to circulate or flow and mix with the staining reagent 820 that is introduced.

In one example, the cell trapping array 800 may also include an optical analyzer 822. The optical analyzer 822 may be an illuminated focal microscope. The optical analyzer 822 may capture images of the stained cell 814 to analyze the structures inside of the cell 814 that have been stained. The optical analyzer 822 may be on a movable track or rail to allow the optical analyzer 822 to move between different orifices 804. Thus, the cell trapping array 800 may allow for in-situ staining and analysis of the cells 814.

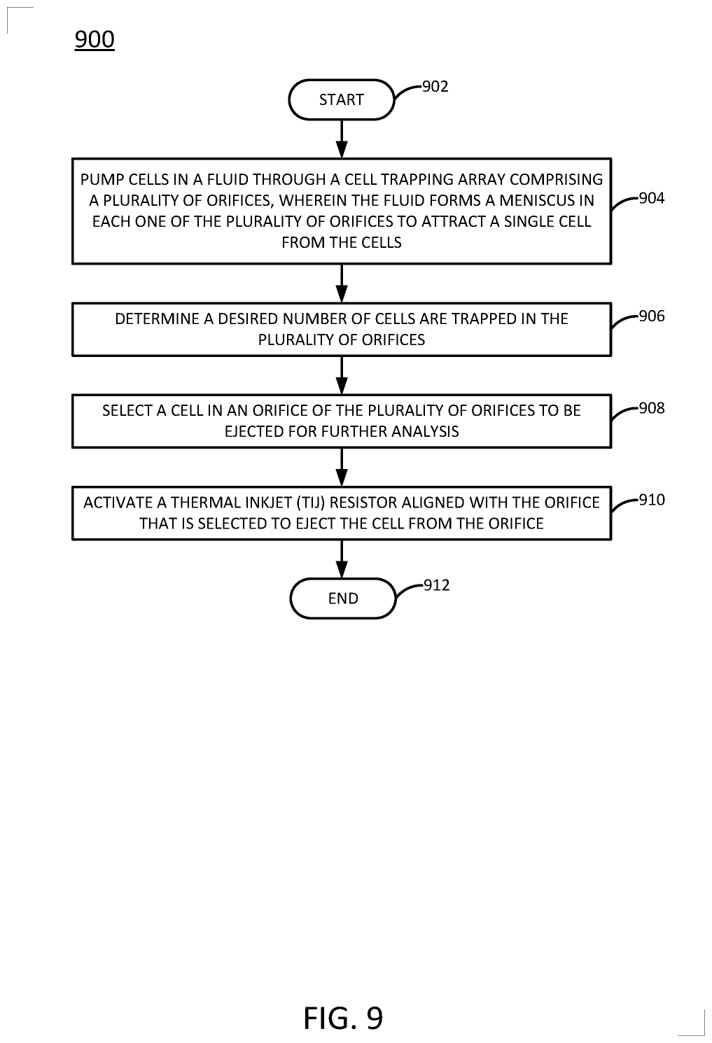

FIG. 9 illustrates a flow diagram of an example method 900 for selectively ejecting cells in a cell trapping array of the present disclosure. In an example, the method 900 may be performed by the cell trapping array 100.

At block 902, the method 900 begins. At block 904, the method 900 pumps cells in a fluid through a cell trapping array comprising a plurality of orifices, wherein the fluid forms a meniscus in each one of the plurality of orifices to attract a single cell from the cells. As described above, the cells can be trapped against a wall of the orifices in accordance with the “cheerio's effect” described above.

In one example, the cells may be drawn towards the orifices as the fluid evaporates. In one example, TIJ resistors may be used to help direct the cells towards the meniscus in the orifices. The TIJ resistors may be activated to create a steam bubble that may move towards the orifice. When the cell is located between the TIJ resistor and the orifice, the TIJ resistor may be activated to create the steam bubble that pushes the cell towards the meniscus in the orifice.

In one example, the orifice may include electrodes to assist in trapping the cells in the orifices. The electrodes may be activated to control a shape of the meniscus and control a rate of capture of the cells. The electrodes may be used with the TIJ resistors to help capture or trap the cells in the orifices.

In one example, the cell concentration may include different types or sizes of cells. The orifices may be the same size or different sizes to trap the same type of cells or different types of cells. The cell concentration may be pumped through a recirculation loop or different recirculation loops from a reservoir that contains the cell concentration.

At block 906, the method 900 determines that a desired number of cells are trapped in the plurality of orifices. In one example, the cell concentration may be continuously pumped through the cell trapping array until a desired number of cells from the cell concentration is trapped. In one example, an optical analyzer may be coupled to the reservoir that contains the cell concentration. The cell concentration in the reservoir can be periodically examined and analyzed to calculate the cell concentration. When the cell concentration falls below a threshold in the reservoir, it may be determined that the desired number of cells have been trapped in the plurality of orifices.

In an example, an optical system (e.g., a camera) may be used to visually see when a cell is trapped in the plurality of orifices. In another example, a sensor may be located in the orifice to indicate the presence of a cell trapped in the orifice. The optical system or sensor may also be used to determine that the desired number of cells have been trapped in the plurality of orifices.

At block 908, the method 900 selects a cell in an orifice of the plurality of orifices to be ejected for further analysis. As noted above, the cell trapping arrays of the present disclosure may include a selective ejection system. The selective ejection system may allow cells in a selected orifice to be ejected. In other words, the selective ejection system allows for one cell to be ejected at a time from a respective orifice rather than having all the cells ejected at the same time.

In one example, the cell for ejection may be selected via a user interface. For example, the user interface may provide information related to which cell is trapped in which orifice. An optical system (e.g., a camera) may provide a visual of which orifices contain a cell. In another example, a sensor may be located in the orifice to indicate that a cell is trapped. The user interface may indicate which orifices contain a cell. The user may then select the cell to be ejected for further analysis.

At block 910, the method 900 activates a thermal inkjet (TIJ) resistor aligned with the orifice that is selected to eject the cell from the orifice. In one example, the selective ejection system may include an array of TIJ resistors. The TIJ resistors may be activated to create a steam bubble. The steam bubble may burst when it reaches the meniscus of the fluid that releases energy. The energy may be enough to overcome the “cheerio's effect” and release the cell out of the orifice.

In one example, the cell may be ejected into an adjacent fluidic channel to create an emulsion for further analysis. As described above, the adjacent fluidic channel may include a different fluid that is immiscible with the fluid containing the cell concentration. In one example, the adjacent fluidic channel may contact the fluid containing the cell concentration. In another example, an air interface may be located between the fluid containing the cell concentration and the adjacent fluidic channel. At block 912, the method 900 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A cell trapping array, comprising:
a plurality of plates coupled along adjacent edges to form a channel;
a plurality of orifices formed in a first plate of the plurality of plates of the channel, wherein the plurality of orifices is shaped to create a meniscus of a fluid in the channel in the plurality of orifices that is to attract a single cell from cells flowing through the channel in the fluid; and
a selective ejection system coupled to a second plate located opposite the first plate of the channel, wherein the selective ejection system is to selectively eject the single cell from one of the plurality of orifices.

2. The cell trapping array of claim 1, wherein a diameter each one of the plurality of orifices is a function of a size of the single cell to be trapped via a meniscus of the fluid formed in the plurality of orifices.

3. The cell trapping array of claim 1, wherein the diameter comprises approximately 10 microns to 100 microns.

4. The cell trapping array of claim 1, wherein the channel comprises a loop and the cell trapping array, further comprise:
a reservoir coupled to the loop of the channel; and
a pump to circulate the cells in the fluid through the channel until a desired number of cells are trapped in the plurality of orifices.

5. The cell trapping array of claim 4, wherein the pump comprises a reversible pump to allow the cells in the fluid to flow in either direction through the channel.

6. The cell trapping array of claim 1, further comprising:
a dispenser to jet a stain at the single cell in a selected one of the plurality of orifices; and
an imaging optic to analyze the single cell that is stained.

7. The cell trapping array of claim 1, further comprising:
a reservoir containing a staining reagent, wherein the staining reagent is released into the fluid of the cell trapping array to mix with the single cell in each one of the plurality of orifices; and
an imaging optic to analyze the single cell that is stained.

8. A cell trapping array, comprising:
a plurality of plates coupled along adjacent edges to form a channel;
a plurality of orifices formed in a first plate of the plurality of plates of the channel, wherein the plurality of orifices is shaped to create a meniscus of a fluid in the channel in the plurality of orifices that is to attract a single cell from cells flowing through the channel in the fluid; and
a thermal inkjet (TIJ) resistor array coupled to a second plate located opposite the first plate of the channel, wherein the TIJ resistor array is to selectively eject the single cell from one of the plurality of orifices.

9. The cell trapping array of claim 8, wherein each one of the plurality of orifices comprises an electrode, wherein the electrode is activated to control a shape of the meniscus in a respective orifice.

10. The cell trapping array of claim 8, wherein the channel comprises a plurality of barriers to control a flow of the cells in the fluid below the plurality of orifices.

11. The cell trapping array of claim 8, wherein the TIJ resistor array comprises a TIJ resistor for each one of the plurality of orifices.

12. The cell trapping array of claim 11, wherein the TIJ resistor for each one of the plurality of orifices is located below and aligned with a respective orifice.

13. A method, comprising:
pumping cells in a fluid through a cell trapping array comprising a plurality of orifices, wherein the fluid forms a meniscus in each one of the plurality of orifices to attract a single cell from the cells;
determining a desired number of cells are trapped in the plurality of orifices;
selecting a cell in an orifice of the plurality of orifices to be ejected for further analysis; and
activating a thermal inkjet (TIJ) resistor aligned with the orifice that is selected to eject the cell from the orifice.

14. The method of claim 13, wherein the cell is ejected into an adjacent fluidic channel.

15. The method of claim 13, further comprising:
activating an electrode in the plurality of orifices to control a shape of the meniscus in accordance with a desired rate of capture.
